SEQUENCE LISTING (2 sequences)

SEQ ID NO: 1 - protein, 7 aa, Artificial sequence
  synthetic
KGGRAKD

SEQ ID NO: 2 - protein, 7 aa, Artificial sequence
  synthetic
DKARGGK

(19) United States
(12) Patent Application Publication
(10) Pub. No.: US 2024/0238362 A1
(43) Pub. Date: Jul. 18, 2024

(54) ANTI-OBESITY PEPTIDES AND USES THEREOF

(71) Applicant: Arizona Board of Regents on Behalf of the University of Arizona, Tucson, AZ (US)

(72) Inventors: Youngwook Won, Tucson, AZ (US); David Bull, Tucson, AZ (US); Minjin Jung, Tucson, AZ (US); Angela Kim, Tucson, AZ (US)

(21) Appl. No.: 17/917,618

(22) PCT Filed: Apr. 6, 2021
(86) PCT No.: PCT/US21/25973

§ 371 Date: Oct. 7, 2022

Related U.S. Application Data
(60) Provisional application No. 63/006,119, filed on Apr. 7, 2020.

(51) Int. Cl. A61K 38/08; A61K 38/06; A61P 3/04

(52) U.S. Cl. CPC A61K 38/08; A61K 38/06; A61P 3/04

(57) ABSTRACT

Provided herein are compositions and methods for treating obesity. In particular, provided herein are anti-obesity peptides and uses of such peptides in the treatment and prevention of obesity and related medical conditions. Further disclosed are the sequences of anti-obesity peptides, wherein the peptides can be cyclized via the addition of a cysteine to each end of the said peptides.

### CROSS REFERENCE TO RELATED APPLICATION

This application claims the benefit of U.S. Prov. Appl. 63/006,119, filed Apr. 7, 2020 which is incorporated by reference herein in its entirety.

### FIELD

Provided herein are compositions and methods for treating obesity. In particular, provided herein are anti-obesity peptide and uses of such peptides in the treatment and prevention of obesity and related medical conditions.

### BACKGROUND

In 2015, more than one in three adults in the US (approximately 116 million adults) were found to suffer from obesity (Stevens, J., et al., Obesity (Silver Spring) 23, 527-531, (2015)). This patient population, which fits the criteria for use of anti-obesity medications, is almost four times as large as the diabetes population. Interestingly, patients visit a pharmacy 15 times more frequently for antidiabetes medications than for anti-obesity medications.

People struggling with being overweight first attempt to change their eating habits and increase physical activity. It is hard for patients to maintain this unaccustomed lifestyle long-term. Also, many patients still believe that obesity does not require pharmacological treatments and they can achieve meaningful weight loss through behavioral changes alone. There are a number of reasons why people avoid taking these anti-obesity medications, even though five FDA-approved medications currently exist (Prescription Medications to Treat Overweight and Obesity, niddk.nih.gov/healthinformation/weight-management/prescription-medications-treat-overweight-obesity). The most important reason is that primary care providers may be uncomfortable prescribing these agents due to the possible side effects that are consequences of the off-targeted mechanism of action of these drugs (Elangbam, C. S. Veterinary pathology 46, 10-24, (2009); Cooke, D. & Bloom, S. T, Nature reviews. Drug discovery 5, 919-931, (2006); Nawrocki, A. R. & Scherer, P. E. Drug discovery today 10, 1219-1230, (2005)). Anti-obesity drugs on the market, Lorcaserin, Phentermine/Topiramate, Naltrexone/Bupropion, Liraglutide, and Orlistat, and most agents currently under development are directed at reducing energy or food intake by acting on the gastrointestinal (GI) tract or the central nervous system (CNS) to control appetite. These drugs have shown severe side effects because the agents exert their activity in wrong organs, tissues, or systems.

Additional therapies for treating and preventing obesity and excess weight are needed.

### SUMMARY

Lipid accumulation in adipose tissue triggers the conversion of adipocytes into disease-stage adipocytes, which have distinctive characteristics at cellular and molecular levels. These diseased adipocytes present certain bio-active molecules on their surface, such as prohibitin, which can be utilized in the development of adipocyte-targeted therapies. Prohibitin is associated with lipid transport in obese adipocytes and adipogenesis (Ande, S. R., et al., Int J Obes (Lond) 36, 1236-1244, (2012); Artal-Sanz, M. & Tavernarakis, N. Nature 461, 793-797, (2009); Brasaemle, D. L., et al., J Biol Chem 279, 46835-46842, (2004)).

A cyclic peptide, C-KGGRAKD-C(SEQ ID NO:1), is a prohibitin-binding peptide (also known as adipocyte-targeting sequence, ATS) (Won, Y. W. et al. Nat Mater 13, 1157-1164, (2014); Kolonin, M. G., et al., Nat Med 10, 625-632, (2004); Patel, N. et al. Proceedings of the National Academy of Sciences of the United States of America 107, 2503-2508, (2010)). Among the 7-11 amino acids in the cyclic peptide, only 2-4 amino acids are involved in the actual binding to the receptor. Accordingly, provided herein are 3 amino acid-long fragments of the ATS peptide. Five different peptide fragments (KGG, GGR, GRA, RAK, and AKD) and a scrambled RKG peptide were generated based on the ATS (KGGRAKD(SEQ ID NO:1)) sequence. Three peptide fragments (GGR, RAK, and AKD) that are able to reduce lipid contents in mature Adipocytes were identified.

The short peptides described herein have the advantage of serving as an oral anti-obesity agent because short peptides can be absorbed spontaneously through the intestinal lumen, while maintaining their stability and structure in acidic conditions in the stomach (Matthews, D. M., et al., Clin Sci 35, 415-424 (1968); Mathews, D. M. & Adibi, S. A. Gastroenterology 71, 151-161 (1976); Craft, I. L., et al., Gut 9, 425-437 (1968)). In experiments described herein each short peptide was given to diet-induced obese (DIO) mice through subcutaneous injections, intraperitoneal injections, or oral (gavage) feeding. Effective prevention of obesity progression in DIO mice given these short peptides by gavage and the degree of prevention by oral delivery was comparable to the injection routes.

Accordingly, provided herein is a pharmaceutical, dietary supplement, or nutraceutical composition comprising one or more (e.g., a combination of two or more, three or more, etc. different) peptides having an amino acid sequence selected from the, for example, KGGRAKD (SEQ ID NO:1), KGG, GRA, GGR, RAK, AKD, DKA, KAR, RGG, ARG, GGK, or DKARGGK (SEQ ID NO:2) or a variant or mimetic thereof. In some embodiments, the composition comprises KGGRAKD (SEQ ID NO:1) and one or more of KGG, GRA, GGR, RAK, or AKD. In some embodiments, the peptide is cyclized (e.g., via the addition of a cysteine to each end of the peptide). In certain embodiments, the peptide is modified. In some embodiments, the composition comprises a pharmaceutically-acceptable carrier. In some embodiments, the composition further comprises one or more additional therapeutic agents (e.g., anti-obesity agents).

The present disclosure is not limited to a particular formulation. The compositions described herein can be formulated for oral, parenteral, or other delivery means. In some exemplary embodiments, the composition is formulated for oral delivery. In some embodiments, the composition is provided as a beverage or food product. In some embodiments, the composition is formulated as a capsule, tablet, or powder.

Additional embodiments provide a composition as described herein for use in treating or preventing obesity.

Further embodiments provide the use of a composition as described herein for treating or preventing obesity.

Yet other embodiments provide the use of a composition as described herein in the preparation of a medicament.

Still other embodiments provide a method of treating or preventing obesity, comprising: administering a composition as described herein to a subject in need thereof. In some embodiments, the administering decreases fat storage and/or promotes weight loss in the subject. In some embodiments, the subject is overweight, obese, or morbidly obese. In some embodiments, the subject has an obesity-related complication.

Additional embodiments are described herein.

### BRIEF DESCRIPTION OF THE DRAWINGS

FIG. 1A: ATS/prohibitin binding determined by immunoprecipitation and western blot. FIG. 1B: Location and degree of prohibitin expression in pre- or mature adipocytes. PM, plasma membrane; Cyt, Cytoplasm. C: Confocal micrographs showing the location of prohibitin in mature- or pre-adipocytes.

FIG. 2A-G: Representative photomicrographs showing Oil Red O-stained mature adipocytes treated with peptides at 100 μM (Day 21). H: Quantitative analysis of the lipids accumulated in the adipocytes (Day 21). Control absorbance: 0.526 (Red line). I: Relative cell counts on day 21.

FIG. 3A-H: IP Preobese Body Weight. Relative body weight measurements for IP injected pre-obese mice. I-J: Body weight change. Changes in body weight compared using HFD control group as baseline value A1-F1: GTT. Blood glucose levels of peptide groups at endpoint, post-glucose injection (after 16 h fasting). A2-F2: ITT. Blood glucose levels of peptide groups at endpoint, post-insulin injection (after 6 h fasting).

FIG. 4A-F: SC Pre-obese Body Weight. Relative body weight measurements for SC injected pre-obese mice. G-H: Body weight change. Changes in body weight compared using HFD control group as baseline value. A1-F1: GTT. Blood glucose levels of peptide groups at endpoint, post-glucose injection (after 16 h fasting). A2-F2: ITT. Blood glucose levels of peptide groups at endpoint, post-insulin injection (after 6 h fasting).

FIG. 5A-F: IP Post-obese Body Weight. Relative body weight measurements for IP injected post-obese mice. G-H: Body weight change. Changes in body weight compared using HFD control group as baseline value. A1-F1: GTT. Blood glucose levels of peptide groups at endpoint, post-glucose injection (after 16 h fasting). A2-F2: ITT. Blood glucose levels of peptide groups at endpoint, post-insulin injection (after 6 h fasting).

FIG. 6A-F: SC Post-obese Body Weight. Relative body weight measurements for SC injected post-obese mice. G-H: Body weight change. Changes in body weight compared using HFD control group as baseline value. A1-D1,F1: GTT. Blood glucose levels of peptide groups at endpoint, post-glucose injection (after 16 h fasting). A2-D2,F2: ITT. Blood glucose levels of peptide groups at endpoint, post-insulin injection (after 6 h fasting).

FIG. 7A-F: Oral/Feeding Pre-obese Body Weight. Relative body weight measurements for orally administered pre-obese mice. A1-F1: GTT. Blood glucose levels at endpoint, post-glucose injection (after 16 h fasting). A2-F2: ITT. Blood glucose levels at endpoint, post-insulin injection (after 6 h fasting).

FIG. 8A-F: Oral/Feeding Post-obese Body Weight. Relative body weight measurements for orally administered post-obese mice. A1-F1: GTT. Blood glucose levels at endpoint, post-glucose injection (after 16 h fasting). A2-F2: ITT. Blood glucose levels at endpoint, post-insulin injection (after 6 h fasting).

FIG. 9. Histological analysis fat pads. An automated system detected and counted adipocytes (yellow line). Adipocyte number and size: average of 3 hpf images from 2 mice (total 6 images/group).

### Definitions

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide” is a reference to one or more peptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, the term “mutant peptide” or “variant peptide” refers to a peptide having a distinct amino acid sequence from the most common variant occurring in nature, referred to as the “wild-type” sequence. A mutant peptide may be a subsequence of a mutant protein or polypeptide (e.g., a subsequence of a naturally-occurring protein that is not the most common sequence in nature) or may be a peptide that is not a subsequence of a naturally occurring protein or polypeptide.

As used herein, the term “artificial peptide” or “artificial polypeptide” refers to a peptide or polypeptide having a distinct amino acid sequence from those found in natural peptides and/or proteins. An artificial protein is not a subsequence of a naturally occurring protein, either the wild-type (i.e., most abundant) or mutant versions thereof. For example, an artificial peptide or polypeptide is not a subsequence of naturally occurring protein (e.g., ATS protein). An artificial peptide or polypeptide may be produced or synthesized by any suitable method (e.g., recombinant expression, chemical synthesis, enzymatic synthesis, etc.).

The terms “peptide mimetic” or “peptidomimetic” refer to a peptide-like molecule that emulates a sequence derived from a protein or peptide. A peptide mimetic or peptidomimetic may contain amino acids and/or non-amino acid components. Examples of peptidomimitecs include chemically modified peptides, peptoids (side chains are appended to the nitrogen atom of the peptide backbone, rather than to the α-carbons), β-peptides (amino group bonded to the ß carbon rather than the α carbon), etc.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs. Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a human subject that is being treated for a disease or condition.

As used herein, the term “effective amount” refers to the amount of a sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” means an approach to obtaining a beneficial or intended clinical result. The beneficial or intended clinical result may include alleviation of symptoms, a reduction in the severity of the disease, inhibiting a underlying cause of a disease or condition, steadying diseases in a non-advanced state, delaying the progress of a disease, and/or improvement or alleviation of disease conditions.

As used herein, the term “pharmaceutical composition” refers to the combination of an active agent (e.g., ATS-derived peptide) with a carrier, inert or active, making the composition especially suitable for diagnostic or therapeutic use in vitro, in vivo or ex vivo. The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

### DETAILED DESCRIPTION

Obesity develops when accumulation of fat/lipid in adipocytes exceeds the biochemical process that maintains homeostasis of fat storage (Gao, M. & Liu, D. Discovery medicine 17, 319-328 (2014)). White adipocytes primarily store energy in the form of lipids for the long term (Rosen, E. D. & Spiegelman, B. M. Nature 444, 847-853, (2006); Ouchi, N., et al., Nature reviews. Immunology 11, 85-97, (2011)).

Accumulation of excessive amounts of lipids in white adipocytes enlarges the white adipocytes, which later secrete hormones and inflammatory cytokines (Ouchi, N., et al., Nature reviews. Immunology 11, 85-97, (2011)). Obesity-induced complications, including type 2 diabetes and cardiovascular diseases, later occur as the secreted proteins take effects. New anti-obesity drug development thus should focus on targeting adipocytes rather than the GI tract or the CNS, which is the target of traditional anti-obesity agents. Adipocyte-targeted therapies achieve safe weight loss without suppressing appetite. Prohibitin is primarily located on mitochondria of pre-adipocytes or non-obese adipocytes (Artal-Sanz, M. & Tavernarakis, Trends Endocrinol Metab 20, 394-401, (2009); De Pauw, A., et al., Am J Pathol 175, 927-939, (2009)) but shifts its location to the cell membrane as adipocytes mature (Ande, S. R., et al., Int J Obes (Lond) 36, 1236-1244, (2012); Won, Y. W. et al. Nat Mater 13, 1157-1164, (2014)). The degree of prohibitin expression depends on the extent of adipogenesis (Patel, N. et al. Proceedings of the National Academy of Sciences of the United States of America 107, 2503-2508, (2010)). A high level of prohibitin expression is observed on the cell membrane in enlarged obese adipocytes, whereas moderate prohibitin expression is found in early-stage obese adipocytes. The discrepancies in prohibitin expression in various stages of adipogenesis render it a therapeutic target for obesity.

Accordingly, provided herein are compositions and methods for blocking prohibitin pathways with ATS. Results described herein indicate that a therapeutic lead, RAK peptide, has shown anti obese activity. These peptides exert anti-obese activity by reducing lipid contents in white adipocytes instead of controlling appetite, which is the primary strategy of current anti-obesity agents. The adipocyte-targeted mechanism and utilization of safe oral delivery of peptides make the peptides a useful preventative intervention.

Provided herein are compositions (e.g., ATS peptides, ATS derived peptides, combinations of ATS-derived peptides, etc.) which reduce accumulation of fat and/or reduce or prevent obesity (e.g., in obese individuals).

In some embodiments, provided herein are compositions, kits, systems, and/or methods to treat, prevent, reduce the likelihood, treat/prevent a side effect of one or more of obesity, excess weight, and associated health problems.

In some embodiments, provided herein are compositions comprising (e.g., pharmaceutical or nutritional compositions) ATS peptides or ATS derived peptides (e.g., one (e.g., 1, 2, 3, 4 or 5) or more of KGG, GGR, GRA, RAK, or AKD), nucleic acids encoding the peptides, proteins and polypeptides herein, molecular complexes of the foregoing, etc. for the treatment or prevention of obesity and/or related diseases and conditions. In some embodiments, compositions comprise multiple different peptides (e.g., an ATS peptide and one or more of the described ATS derived peptides). In some embodiments, the peptides are one or more of KGGRAKD (SEQ ID NO:1), KGG, GRA, GGR, RAK, AKD, DKA, KAR, RGG, ARG, GGK, or DKARGGK (SEQ ID NO:2).

In some embodiments, a peptide provided herein is an artificial, not naturally-occurring, sequence. In some embodiments, a peptide described herein is prepared by methods known to those of ordinary skill in the art. For example, the peptide can be synthesized using solid phase polypeptide synthesis techniques (e.g. Fmoc or Boc chemistry). Alternatively, the peptide can be produced using recombinant DNA technology (e.g., using bacterial or eukaryotic expression systems). Further, a peptide may be expressed within a subject (e.g., following administration of an appropriate vector). Accordingly, to facilitate such methods, provided herein are genetic vectors (e.g., plasmids, viral vectors (e.g. AAV), etc.) comprising a sequence encoding the peptide, as well as host cells comprising such vectors. Furthermore, provided herein are the peptide produced via such methods.

In some embodiments, the administration of compositions described herein (e.g., ATS and ATS-derived peptides), variants and mimetics thereof, nucleic acids encoding such peptides, etc.) is provided. In some embodiments, provided herein is the administration of bioactive agents which reduce fat accumulation and/or treat obesity in vivo, or are otherwise described herein.

Embodiments are not limited to the specific sequences listed herein. In some embodiments, peptides meeting limitations described herein and having substitutions not explicitly described are within the scope of embodiments here. In some embodiments, the peptides described herein are further modified (e.g., substitution, deletion, or addition of standard amino acids; chemical modification; etc.). Modifications that are understood in the field include N-terminal modification, C-terminal modification (which protects the peptide from proteolytic degradation), alkylation of amide groups, hydrocarbon “stapling” (e.g., to stabilize conformations). In some embodiments, the peptides/polypeptides described herein may be modified by conservative residue substitutions, for example, of the charged residues (K to R, R to K, D to E and E to D). Modifications of the terminal carboxy group include, without limitation, the amide, lower alkyl amide, constrained alkyls (e.g. branched, cyclic, fused, adamantyl) alkyl, dialkyl amide, and lower alkyl ester modifications. Lower alkyl is C1-C4 alkyl. Furthermore, one or more side groups, or terminal groups, may be protected by protective groups known to the ordinarily-skilled peptide chemist. The α-carbon of an amino acid may be mono- or dimethylated.

In some embodiments, peptides are provided comprising: (i) one or more of the amino acid residues in the peptide are D-enantiomers, (ii) an N-terminally acetyl group, (iii) a deamidated C-terminal group, (iv) one or more unnatural amino acids, (v) one or more amino acid analogs, and/or (vi) one or more peptoid amino acids. In some embodiments, the peptide or an amino acid therein comprises a modification selected from the group consisting of phosphorylation, glycosylation, ubiquitination, S-nitrosylation, methylation, N-acetylation, lipidation, lipoylation, deimination, eliminylation, disulfide bridging, isoaspartate formation, racemization, glycation; carbamylation, carbonylation, isopeptide bond formation, sulfation, succinylation, S-sulfonylation, S-sulfinylation, S-sulfenylation, S-glutathionylation, pyroglutamate formation, propionylation, adenylylation, nucleotide addition, iodination, hydroxylation, malonylation, butyrylation, amidation, C-terminal amidation, de-amidation, alkylation, acylation, biotinylation, carbamylation, oxidation, and pegylation.

In some embodiments, any embodiments described herein may comprise mimetics corresponding to ATS-derived peptide and/or variants thereof, with various modifications that are understood in the field. In some embodiments, residues in the peptide sequences described herein may be substituted with amino acids having similar characteristics (e.g., hydrophobic to hydrophobic, neutral to neutral, etc.) or having other desired characteristics (e.g., more acidic, more hydrophobic, less bulky, more bulky, etc.). In some embodiments, non-natural amino acids (or naturally-occurring amino acids other than the standard 20 amino acids) are substituted in order to achieve desired properties.

In some embodiments, residues having a side chain that is positively charged under physiological conditions, or residues where a positively-charged side chain is desired, are substituted with a residue including, but not limited to: lysine, homolysine, δ-hydroxylysine, homoarginine, 2,4-diaminobutyric acid, 3-homoarginine, D-arginine, arginal (—COOH in arginine is replaced by —CHO), 2-amino-3-guanidinopropionic acid, nitroarginine (N(G)-nitroarginine), nitrosoarginine (N(G)-nitrosoarginine), methylarginine (N-methyl-arginine), ε-N-methyllysine, allo-hydroxylysine, 2,3-diaminopropionic acid, 2,2′-diaminopimelic acid, ornithine, sym-dimethylarginine, asym-dimethylarginine, 2,6-diaminohexinic acid, p-aminobenzoic acid and 3-aminotyrosine and, histidine, 1-methylhistidine, and 3-methylhistidine.

A neutral residue is a residue having a side chain that is uncharged under physiological conditions. A polar residue preferably has at least one polar group in the side chain. In some embodiments, polar groups are selected from hydroxyl, sulfhydryl, amine, amide and ester groups or other groups which permit the formation of hydrogen bridges.

In some embodiments, residues having a side chain that is neutral/polar under physiological conditions, or residues where a neutral side chain is desired, are substituted with a residue including, but not limited to: asparagine, cysteine, glutamine, serine, threonine, tyrosine, citrulline, N-methylserine, homoserine, allo-threonine and 3,5-dinitro-tyrosine, and β-homoserine.

Residues having a non-polar, hydrophobic side chain are residues that are uncharged under physiological conditions, preferably with a hydropathy index above 0, particularly above 3. In some embodiments, non-polar, hydrophobic side chains are selected from alkyl, alkylene, alkoxy, alkenoxy, alkylsulfanyl and alkenylsulfanyl residues having from 1 to 10, preferably from 2 to 6, carbon atoms, or aryl residues having from 5 to 12 carbon atoms. In some embodiments, residues having a non-polar, hydrophobic side chain are, or residues where a non-polar, hydrophobic side chain is desired, are substituted with a residue including, but not limited to: leucine, isoleucine, valine, methionine, alanine, phenylalanine, N-methylleucine, tert-butylglycine, octylglycine, cyclohexylalanine, β-alanine, 1-aminocyclohexylcarboxylic acid, N-methylisoleucine, norleucine, norvaline, and N-methylvaline.

In some embodiments, peptides are cyclized (e.g., via cysteine residues on each end of the peptide).

In some embodiments, peptide and polypeptides are isolated and/or purified (or substantially isolated and/or substantially purified). Accordingly, in such embodiments, peptides and/or polypeptides are provided in substantially isolated form. In some embodiments, peptides and/or polypeptides are isolated from other peptides and/or polypeptides as a result of solid phase peptide synthesis, for example. Alternatively, peptides and/or polypeptides can be substantially isolated from other proteins after cell lysis from recombinant production. Standard methods of protein purification (e.g., HPLC) can be employed to substantially purify peptides and/or polypeptides. In some embodiments, the present invention provides a preparation of peptides and/or polypeptides in a number of formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations may contain protective agents, such as buffers, preservatives, cryprotectants (e.g., sugars such as trehalose), etc. The form of such preparations can be solutions, gels, etc. In some embodiments, peptides and/or polypeptides are prepared in lyophilized form. Moreover, such preparations can include other desired agents, such as small molecules or other peptides, polypeptides or proteins. Indeed, such a preparation comprising a mixture of different embodiments of the peptides and/or polypeptides described here may be provided.

In some embodiments, provided herein are peptidomimetic versions of the peptide sequences described herein or variants thereof. In some embodiments, a peptidomimetic is characterized by an entity that retains the polarity (or non-polarity, hydrophobicity, etc.), three-dimensional size, and functionality (bioactivity) of its peptide equivalent but wherein all or a portion of the peptide bonds have been replaced (e.g., by more stable linkages). In some embodiments, ‘stable’ refers to being more resistant to chemical degradation or enzymatic degradation by hydrolytic enzymes. In some embodiments, the bond which replaces the amide bond (e.g., amide bond surrogate) conserves some properties of the amide bond (e.g., conformation, steric bulk, electrostatic character, capacity for hydrogen bonding, etc.). Cyclization (head-to-tail, head/tail-to-side-chain, and/or side-chain-to-side-chain) enhances peptide stability and permeability by introducing conformation constraint, thereby reducing peptide flexibility, and a cyclic enkephalin analog is highly resistant to enzymatic degradation. Chapter 14 of “Drug Design and Development”, Krogsgaard, Larsen, Liljefors and Madsen (Eds) 1996, Horwood Acad. Publishers provides a general discussion of techniques for the design and synthesis of peptidomimetics and is herein incorporated by reference in its entirety. Suitable amide bond surrogates include, but are not limited to: N-alkylation (Schmidt, R. et al., Int. J. Peptide Protein Res., 1995, 46, 47; herein incorporated by reference in its entirety), retro-inverse amide (Chorev, M. and Goodman, M., Acc. Chem. Res, 1993, 26, 266; herein incorporated by reference in its entirety), thioamide (Sherman D. B. and Spatola, A. F. J. Am. Chem. Soc., 1990, 112, 433; herein incorporated by reference in its entirety), thioester, phosphonate, ketomethylene (Hoffman, R. V. and Kim, H. O. J. Org. Chem., 1995, 60, 5107; herein incorporated by reference in its entirety), hydroxymethylene, fluorovinyl (Allmendinger, T. et al., Tetrahydron Lett., 1990, 31, 7297; herein incorporated by reference in its entirety), vinyl, methyleneamino (Sasaki, Y and Abe, J. Chem. Pharm. Bull. 1997 45, 13; herein incorporated by reference in its entirety), methylenethio (Spatola, A. F., Methods Neurosci, 1993, 13, 19; herein incorporated by reference in its entirety), alkane (Lavielle, S. et. al., Int. J.Peptide Protein Res., 1993, 42, 270; herein incorporated by reference in its entirety) and sulfonamido (Luisi, G. et al. Tetrahedron Lett. 1993, 34, 2391; herein incorporated by reference in its entirety).

As well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Suitable peptidomimetics include reduced peptides where the amide bond has been reduced to a methylene amine by treatment with a reducing agent (e.g. borane or a hydride reagent such as lithium aluminum-hydride); such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142; herein incorporated by reference in its entirety.

In various embodiments, the peptides disclosed herein are derivatized by conjugation to one or more polymers or small molecule substituents.

In certain of these embodiments, the peptides described herein (or variants thereof) are derivatized by coupling to polyethylene glycol (PEG). Coupling may be performed using known processes. See, Int. J. Hematology, 68:1 (1998); Bioconjugate Chem., 6:150 (1995); and Crit. Rev. Therap. Drug Carrier Sys., 9:249 (1992) all of which are incorporated herein by reference in their entirety. Those skilled in the art, therefore, will be able to utilize such well-known techniques for linking one or more polyethylene glycol polymers to the peptides and polypeptides described herein. Suitable polyethylene glycol polymers typically are commercially available or may be made by techniques well known to those skilled in the art. The polyethylene glycol polymers preferably have molecular weights between 500 and 20,000 and may be branched or straight chain polymers.

The attachment of a PEG to a peptide or polypeptide described herein can be accomplished by coupling to amino, carboxyl or thiol groups. These groups will typically be the N- and C-termini and on the side chains of such naturally occurring amino acids as lysine, aspartic acid, glutamic acid and cysteine. Since the peptides and polypeptides of the present disclosure can be prepared by solid phase peptide chemistry techniques, a variety of moieties containing diamino and dicarboxylic groups with orthogonal protecting groups can be introduced for conjugation to PEG.

The present disclosure also provides for conjugation of the peptides described herein (variants thereof) to one or more polymers other than polyethylene glycol.

In some embodiments, the peptides described herein are derivatized by conjugation or linkage to, or attachment of, polyamino acids (e.g., poly-his, poly-arg, poly-lys, etc.) and/or fatty acid chains of various lengths to the N- or C-terminus or amino acid residue side chains. In certain embodiments, the peptides and polypeptides described herein are derivatized by the addition of polyamide chains, particularly polyamide chains of precise lengths, as described in U.S. Pat. No. 6,552,167, which is incorporated by reference in its entirety. In yet other embodiments, the peptides and polypeptides are modified by the addition of alkylPEG moieties as described in U.S. Pat. Nos. 5,359,030 and 5,681,811, which are incorporated by reference in their entireties.

In select embodiments, the peptides described herein (or variants thereof) are derivatized by conjugation to polymers that include albumin and gelatin. See, Gombotz and Pettit, Bioconjugate Chem., 6:332-351, 1995, which is incorporated herein by reference in its entirety.

In further embodiments, the peptides described herein are conjugated or fused to immunoglobulins or immunoglobulin fragments, such as antibody Fc regions.

In some embodiments, the pharmaceutical compositions described herein (e.g., comprising the peptides described herein find use in the treatment and/or prevention of obesity. In some embodiments, the compositions are administered to a subject. In certain embodiments, the patient is an adult. In other embodiments, the patient is a child.

In various embodiments, the peptides described herein are administered in an amount, on a schedule, and for a duration sufficient to decrease body weight of a subject.

In some embodiments, provided herein are pharmaceutical compositions comprising of one or more ATS or ATS derived peptides or variants thereof and a pharmaceutically acceptable carrier. Any carrier which can supply an active peptide or polypeptide (e.g., without destroying the peptide or polypeptide within the carrier) is a suitable carrier, and such carriers are well known in the art. In some embodiments, compositions are formulated for administration by any suitable route, including but not limited to, orally (e.g., such as in the form of tablets, capsules, granules or powders), sublingually, bucally, parenterally (such as by subcutaneous, intravenous, intramuscular, intradermal, or intracisternal injection or infusion (e.g., as sterile injectable aqueous or non-aqueous solutions or suspensions, etc.)), nasally (including administration to the nasal membranes, such as by inhalation spray), topically (such as in the form of a cream or ointment), transdermally (such as by transdermal patch), rectally (such as in the form of suppositories), etc.

The present disclosure is not limited to a particular formulation comprising one or more of the above-described compositions. In some embodiments, compositions are provided as one or more of supplements, food products, foods, and food additives. In some embodiments, foods and food products are one or more of bars (e.g., raw bars), biscuits, crackers, chips, pastes, gruels and liquids beverages, powders, and the like.

In some embodiments, compositions are provided as powders or pastes that can be mixed with a liquid to provide a beverage.

The dietary supplement may comprise one or more inert ingredients, especially if it is desirable to limit the number of calories added to the diet by the dietary supplement. For example, the dietary supplement may also contain optional ingredients including, for example, herbs, vitamins, minerals, enhancers, colorants, sweeteners, flavorants, inert ingredients, and the like.

In further embodiments, the compositions comprise at least one food flavoring such as acetaldehyde, acetoin (acetyl methylcarbinol), anethole (parapropenyl anisole), benzaldehyde (benzoic aldehyde), N butyric acid (butanoic acid), d or l carvone (carvol), cinnamaldehyde (cinnamic aldehyde), citral (2,6 dimethyloctadien 2,6 al 8, gera nial, neral), decanal (N decylaldehyde, capraldehyde, capric aldehyde, caprinaldehyde, aldehyde C 10), ethyl acetate, ethyl butyrate, 3 methyl 3 phenyl glycidic acid ethyl ester (ethyl methyl phenyl glycidate, strawberry aldehyde, C 16 aldehyde), ethyl vanillin, geraniol (3,7 dimethyl 2,6 and 3,6 octadien 1 ol), geranyl acetate (geraniol acetate), limonene (d, 1, and dl), linalool (linalol, 3,7 dimethyl 1,6 octadien 3 ol), linalyl acetate (bergamol), methyl anthranilate (methyl 2 aminobenzoate), piperonal (3,4 methylenedioxy benzaldehyde, heliotropin), vanillin, alfalfa (Medicago sativa L.), allspice (Pimenta officinalis), ambrette seed (Hibiscus abelmoschus), angelic (Angelica archangelica), Angostura (Galipea officinalis), anise (Pimpinella anisum), star anise (Illicium verum), balm (Melissa officinalis), basil (Ocimum basilicum), bay (Laurus nobilis), calendula (Calendula officinalis), (Anthemis nobilis), capsicum (Capsicum frutescens), caraway (Carum carvi), cardamom (Elettaria cardamomum), cassia, (Cinnamomum cassia), cayenne pepper (Capsicum frutescens), Celery seed (Apium graveolens), chervil (Anthriscus cerefolium), chives (Allium schoenoprasum), coriander (Coriandrum sativum), cumin (Cuminum cyminum), elder flowers (Sambucus canadensis), fennel (Foeniculum vulgare), fenugreek (Trigonella foenum graecum), ginger (Zingiber officinale), horehound (Marrubium vulgare), horseradish (Armoracia lapathifolia), hyssop (Hyssopus officinalis), lavender (Lavandula officinalis), mace (Myristica fragrans), marjoram (Majorana hortensis), mustard (Brassica nigra, Brassica juncea, Brassica hirta), nutmeg (Myristica fragrans), paprika (Capsicum annuum), black pepper (Piper nigrum), peppermint (Mentha piperita), poppy seed (Papayer somniferum), rosemary (Rosmarinus officinalis), saffron (Crocus sativus), sage (Salvia officinalis), savory (Satureia hortensis, Satureia montana), sesame (Sesamum indicum), spearmint (Mentha spicata), tarragon (Artemisia dracunculus), thyme (Thymus vulgaris, Thymus serpyllum), turmeric (Curcuma longa), vanilla (Vanilla planifolia), zedoary (Curcuma zedoaria), sucrose, glucose, saccharin, sorbitol, mannitol, aspartame. Other suitable flavoring are disclosed in such references as Remington's Pharmaceutical Sciences, 18th Edition, Mack Publishing, p. 1288-1300 (1990), and Furia and Pellanca, Fenaroli's Handbook of Flavor Ingredients, The Chemical Rubber Company, Cleveland, Ohio, (1971), known to those skilled in the art.

In other embodiments, the compositions comprise at least one synthetic or natural food coloring (e.g., annatto extract, astaxanthin, beet powder, ultramarine blue, canthaxanthin, caramel, carotenal, beta carotene, carmine, toasted cottonseed flour, ferrous gluconate, ferrous lactate, grape color extract, grape skin extract, iron oxide, fruit juice, vegetable juice, dried algae meal, tagetes meal, carrot oil, corn endosperm oil, paprika, paprika oleoresin, riboflavin, saffron, tumeric, tumeric and oleoresin).

In some embodiments, provided herein are methods for treating patients suffering from (or at risk of) obesity and related conditions. In some embodiments, a pharmaceutical composition comprising at least one ATS or ATS-derived peptide described herein (or variants thereof) is delivered to such a patient in an amount and at a location sufficient to treat the condition. In some embodiments, peptides (or pharmaceutical composition comprising such) can be delivered to the patient systemically (e.g., orally or parenterally) and it will be within the ordinary skill of the medical professional treating such patient to ascertain the most appropriate delivery route, time course, and dosage for treatment. It will be appreciated that application methods of treating a patient most preferably substantially alleviates or even eliminates such symptoms; however, as with many medical treatments, application of the inventive method is deemed successful if, during, following, or otherwise as a result of the inventive method, the symptoms of the disease or disorder in the patient subside to an ascertainable degree.

A pharmaceutical or nutritional composition may be administered in the form which is formulated with a pharmaceutically acceptable carrier and optional excipients, adjuvants, etc. in accordance with good pharmaceutical practice. The peptide composition may be in the form of a solid, semi-solid or liquid dosage form: such as powder, solution, elixir, syrup, suspension, cream, drops, paste and spray. As those skilled in the art would recognize, depending on the chosen route of administration (e.g. pill, injection, etc.), the composition form is determined. In general, it is preferred to use a unit dosage form in order to achieve an easy and accurate administration of the active pharmaceutical peptide or polypeptide. In general, the therapeutically effective pharmaceutical compound is present in such a dosage form at a concentration level ranging from about 0.5% to about 99% by weight of the total composition, e.g., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, a peptide described herein is provided in a unit dosage form for administration to a subject, comprising one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed. The sterile injectable preparation may employ a nontoxic parenterally acceptable diluent or solvent such as sterile nonpyrogenic water or 1,3-butanediol. Among the other acceptable vehicles and solvents that may be employed are 5% dextrose injection, Ringer's injection and isotonic sodium chloride injection (as described in the USP/NF). In addition, sterile, fixed oils may be conventionally employed as solvents or suspending media. For this purpose, any bland fixed oil may be used, including synthetic mono-, di- or triglycerides. Fatty acids such as oleic acid can also be used in the preparation of injectable compositions.

In certain embodiments, the peptides described herein (or variants thereof) are administered in an amount, expressed as a daily equivalent dose regardless of dosing frequency, of 50 micrograms (“mcg”) per day, 60 mcg per day, 70 mcg per day, 75 mcg per day, 100 mcg per day, 150 mcg per day, 200 mcg per day, or 250 mcg per day. In some embodiments, the peptides described herein (or variants thereof) are administered in an amount of 500 mcg per day, 750 mcg per day, or 1 milligram (“mg”) per day. In yet further embodiments, the peptides described herein are administered in an amount, expressed as a daily equivalent dose regardless of dosing frequency, of 1-10 mg per day, including 1 mg per day, 1.5 mg per day, 1.75 mg per day, 2 mg per day, 2.5 mg per day, 3 mg per day, 3.5 mg per day, 4 mg per day, 4.5 mg per day, 5 mg per day, 5.5 mg per day, 6 mg per day, 6.5 mg per day, 7 mg per day, 7.5 mg per day, 8 mg per day, 8.5 mg per day, 9 mg per day, 9.5 mg per day, or 10 mg per day.

In various embodiments, the peptides described herein (or variants thereof) are administered on a monthly, biweekly, weekly, daily (“QD”), or twice a day (“BID”) dosage schedule. In other embodiments, the peptide/polypeptide is administered. In typical embodiments, the peptide/polypeptide is administered for at least 3 months, at least 6 months, at least 12 months, or more. In some embodiments, peptides described herein (or variants thereof) are administered for at least 18 months, 2 years, 3 years, or more.

### EXPERIMENTAL

### Example 1

### Assay of Peptide Activity in In Vitro Human System

In murine-based in vitro systems, GGR, RAK, and AKD were effective in reducing lipid accumulation. These peptides are expected to play similar roles in human mature adipocytes in inhibiting lipid accumulation and adipogenesis. This example describes testing of lead peptide candidates, GGR, RAK, and AKD, in human adipocytes for improved translational potential. Due to structural similarities between murine and human physiology, interactions of the short peptides with human mature adipocytes are predicted to show similar results in reducing lipid accumulation that were seen in mouse adipocytes.

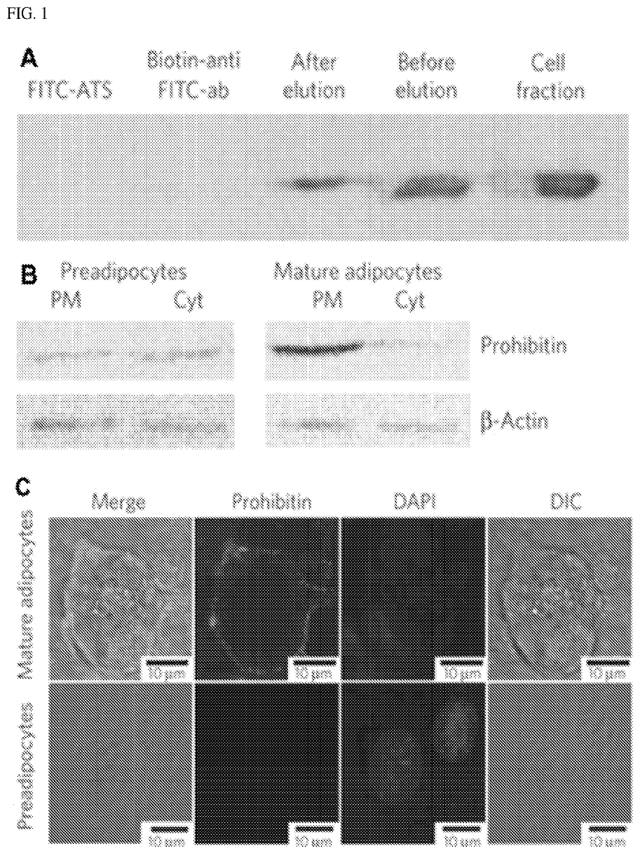

It was confirmed that prohibitin shifts its location to the cell membrane as adipogenesis progresses and ATS binds to prohibition. The ATS/prohibitin binding was verified in pre- or mature adipocytes (Won, Y. W. et al. Nat Mater 13, 1157-1164, (2014)). Fractionated proteins isolated from adipocytes were incubated with the ATS magnetic beads and the protein captured by ATS was identified by western blotting. The bound protein to ATS was revealed to be prohibitin (FIG. 1A). This indicates that ATS specifically binds to prohibitin. Prohibitin is observed in several intracellular locations at varying expression levels depending on the degree of adipogenesis (Wang, P. et al. Cell Mol Life Sci 61, 2405-2417, (2004); Patel, N. et al. Proceedings of the National Academy of Sciences of the United States of America 107, 2503-2508, (2010)). The degree and location of prohibitin expression in pre- or mature adipocytes was determined. More prohibitin is found on the cell membrane (PM) of mature adipocytes, while no difference in prohibitin expression was observed in pre-adipocytes (FIG. 1B). Confocal microscopy also verified the prohibitin expression on the cell membrane of mature adipocytes (FIG. 1C). High-level prohibitin expression on the cell membrane of mature adipocytes is important to the ATS/prohibitin interaction.

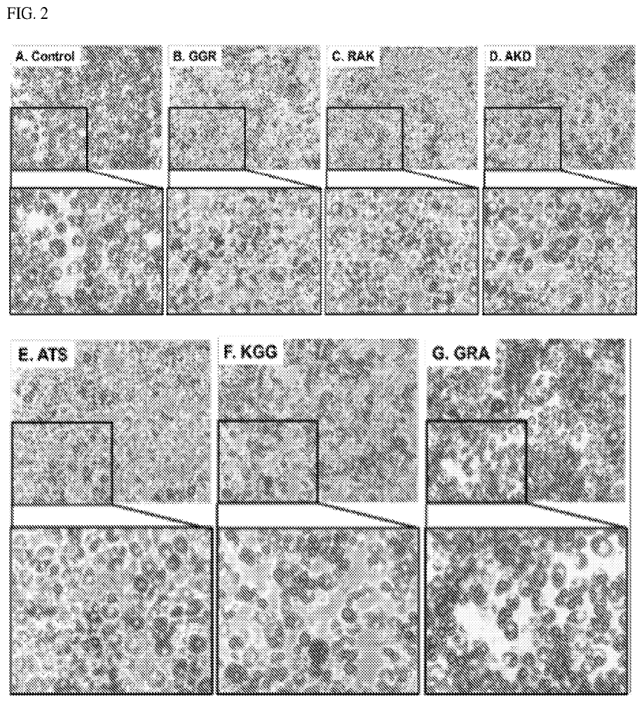

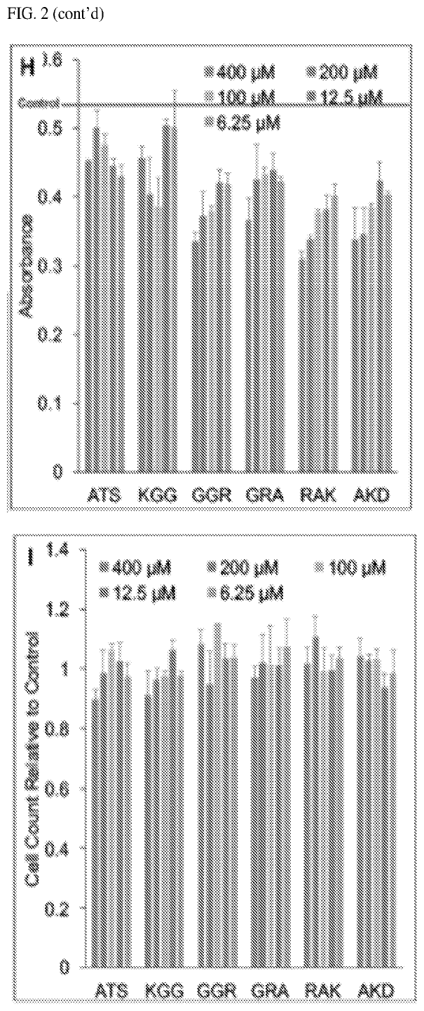

The effects of the minimal short peptides in blocking lipid accumulation in adipocytes were compared with their parent full-length peptide. After generating five different 3 amino acid fragments (KGG, GGR, GRA, RAK, and AKD) from the original ATS peptide (KGGRAKD (SEQ ID NO:1)), each peptide was administered to 3T3-LI pre-adipocytes in five varying concentrations. Peptides were treated to the adipocytes three times per week for three weeks. Oil Red O staining was utilized to assess the lipid contents in mature adipocytes in culture. The morphological characteristics of 3T3-LI mature adipocytes were observed by microscopy. The number and size of lipid droplets in adipocytes treated with GGR, RAK, AKD, or ATS (FIG. 2B-E) were smaller than those in the non-treated control (FIG. 2A), whereas moderate differences in the morphology of lipid droplets were observed between KGG and GRA (FIG. 2F, G) vs. control. Furthermore, the lipids were quantitatively analyzed. This result demonstrates that the degree of lipid accumulation in 3T3-LI mature adipocytes was decreased by approximately 20% upon treatment of GGR, RAK, or AKD (FIG. 2H). At the end point, the cell number was almost the same across the treatment groups, meaning that the reduction in lipid accumulation is not a consequence of toxicity of the peptides (FIG. 2I). The most effective peptide candidates were screened. These results identified GGR, RAK, and AKD as lead peptides.

Effects of GGR, RAK, and AKD on lipid accumulation are validated in human adipocytes. Primary human subcutaneous pre-adipocytes are purchased from American Type Culture Collection (ATCC) to be used in all in vitro studies (ATCC PCS-210-010). The adipocyte differentiation-inducing described above is optimized for the generation of human mature adipocytes from the human pre-adipocytes (Zebisch, K., et al., Anal Biochem 425, 88-90, (2012)). Naive human pre-adipocytes serve as the undifferentiated control. The cells are first thawed into two T75 flasks per 1 million cell vial in basal medium I (BMI), which is composed of DMEM (4.5 g/l glucose) supplemented with 10% FBS and 1% Penicillin Streptomycin (P/S). The media is changed the following day, and on day 3, the cells are seeded in 96-well plates (200 μl/well). On day 4, the media is changed to DMI, which is DMEM containing 10% FBS, 1% P/S, 1 μg/μl insulin, 0.5 mM IBMX, and 0.25 μM Dexamethasone. By this day, the viscosity of the media will have increased due to lipid production by the cells. On day 7, the media is changed to DMII, which is DMEM (4.5 g/l glucose) supplemented with 10% FBS, 1% P/S, and 1 μg/μl insulin. It is expected that intracellular droplets are seen after one week of culture and adipogenesis is observed. At the time of full differentiation, the media is changed back to BMI. For the control pre-adipocytes, the media is replenished on days 3, 5, 7, 8, and 12 (Zebisch et al., supra).

The testing of various peptide concentrations accommodates a dose-dependent effect on differentiation and determines optimal doses for use in in vivo studies. To verify the anti-obese effects of the three peptides in human systems, the same protocol as devised for the in vitro animal cell-based systems is used. Cells are maintained in the BMI culture media until the peptide treatments begin (days 12-14). Five different fragments (KGG, GGR, GRA, RAK, and AKD) and the complete ATS sequence (KGGRAKD (SEQ ID NO:1)) are tested on the adipocytes. Various peptide concentrations are used to elucidate the relationship of dose and efficacy of peptide treatment. A monoclonal antibody (mAb) against prohibitin is used as a positive control (Invitrogen, MA5-12858).

The number of viable cells is counted prior to Oil Red O staining using Cell Counting Kit 8 (Dojindo). This cell viability study allows one to determine potential toxicities of the peptides and compensate for cell number variation-causing artifacts. Oil Red O is dissolved in isopropanol at 3 μg/ml to create a working solution. 12 ml of Oil Red O working solution is mixed with 8 ml dH₂O and incubated for 10 minutes. This solution is then filtered using a 0.2 μm syringe filter. Adipocytes seeded on 96-well plates are washed with PBS twice. 10% formaldehyde is added for initial fixation and the plates are incubated for at least 30 minutes. The formaldehyde is removed and the cells are once again washed twice, this time with dH₂O. 60% isopropanol is added to each well. The plates are incubated for 5 minutes. The isopropanol is removed from the wells and replaced with Oil Red O working solution. The plates are incubated for an additional 10 minutes. The Oil Red O solution is removed and washed four times with dH₂O. Lastly, digital micrographs of the lipid droplets in adipocytes are taken using a light microscope. After the staining protocol, all plates are washed three times with 60% isopropanol. Each time, 5 minutes of gentle rocking was used. The Oil Red O is extracted with 100% isopropanol with gentle rocking for 5 minutes. 200 μl isopropanol per well is used as a background to measure absorbance at 492 nm using a BioTek microplate reader.

### Example 2

### Efficacy of the Peptides in Animal Models

Prior to oral administration, peptides were administered to DIO mice via intraperitoneal (IP) and subcutaneous (SC) injections. This study was divided into two phases:

In the first phase, peptides were tested on pre-obese mice that were being fed high fat diet (HFD) to induce obesity (obesity progression model, OPM). In the second phase, these peptides were given to mice that had already developed obesity (obesity model, OM). GGR and RAK exhibited positive effects on prevention of body weight gain in both OPM and OM mice given IP injections. Scrambled peptide, RKG, did not show meaningful efficacy over the vehicle group (PBS). With SC injections, GGR and RAK were effective in OM mice, while AKD turned out to be the best working peptide in OPM mice. As the activity of these three peptides varies depending on the injection route and the disease status, all three peptides, GGR, RAK, and AKD, were tested by oral administration. In OPM mice given oral administration, RAK and AKD were efficacious over GGR. In OM mice, RAK, GGR, and AKD led to similar levels of obesity prevention.

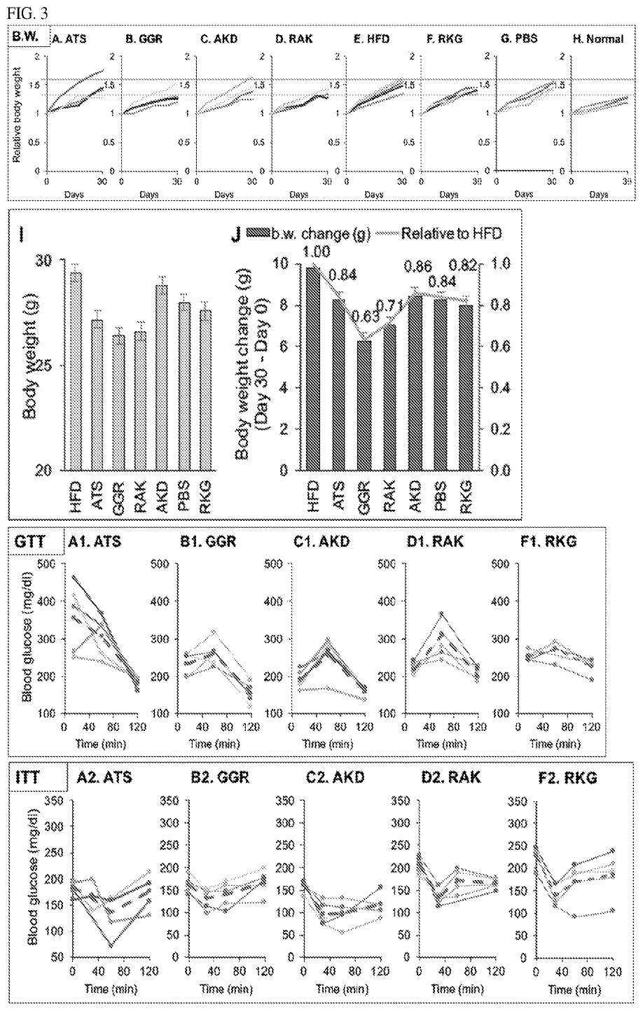

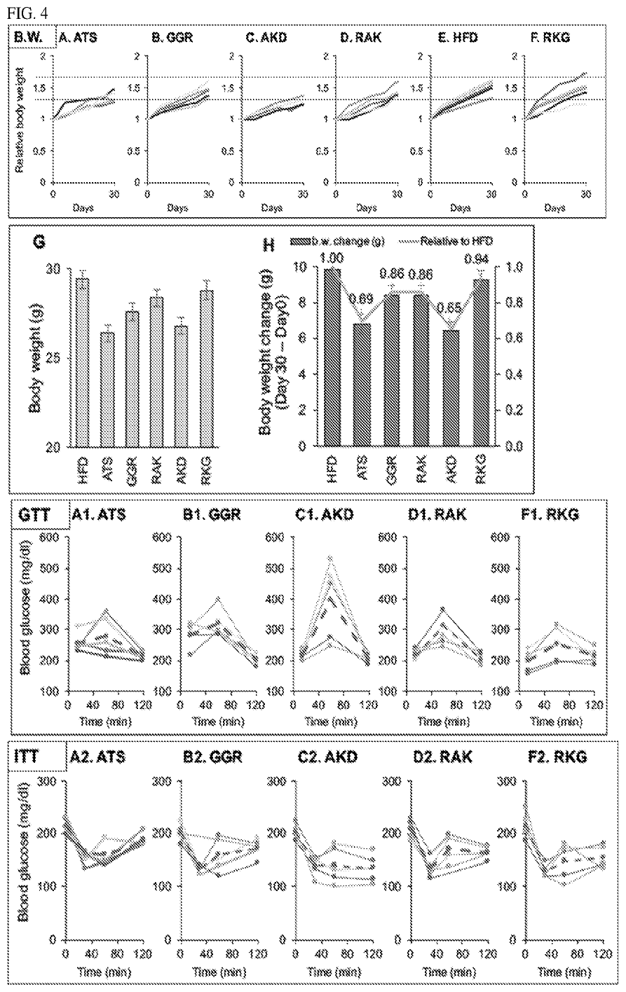

Prior to oral administration, GGR, RAK, AKD, full-length ATS, and scrambled RKG peptides were administered to OPM mice via IP and SC injections to further screen an ideal peptide composition. PBS was included as a vehicle group. GGR, RAK, and AKD peptides were discovered to impede body weight gain in OPM mice. GGR and RAK were effective when given IP injections and AKD was effective by SC injections (FIGS. 3, 4). Scrambled RKG and vehicle showed no anti-obese effects.

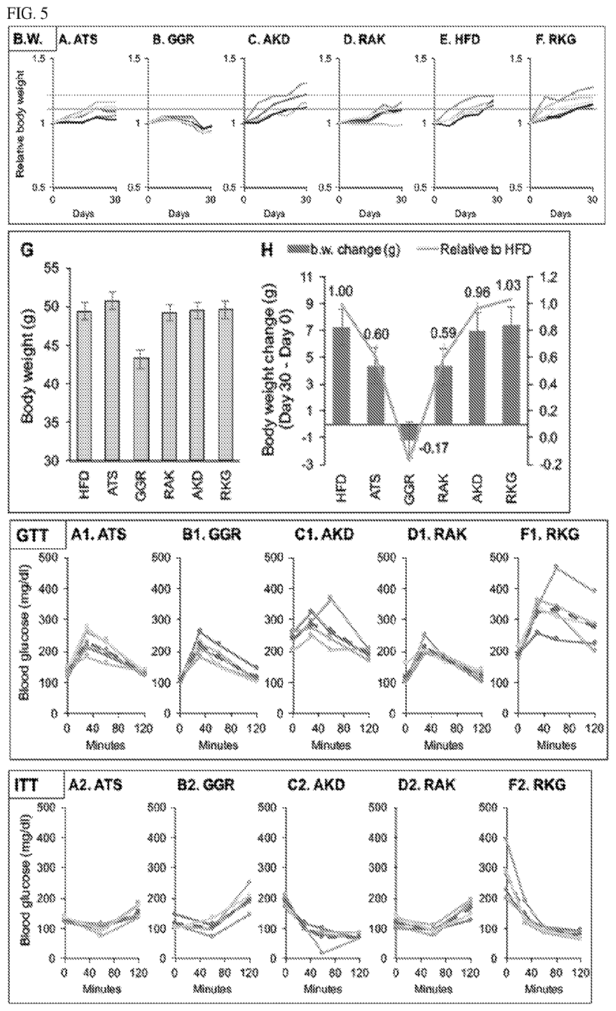

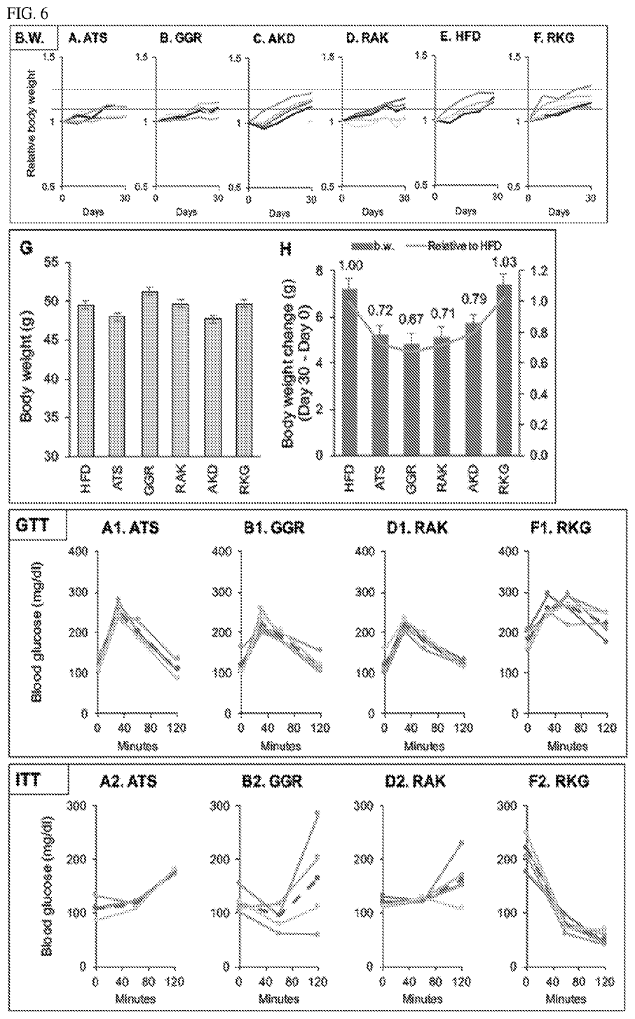

The second phase signified the post-obesity stage, where the same methods from phase 1 were used on OM mice. Body weight change showed that GGR and RAK exhibited similar activity to that of the parent ATS peptide in both injection routes in post-obese mice (FIGS. 5, 6). These results further confirm that GGR and RAK were the most promising peptide compositions.

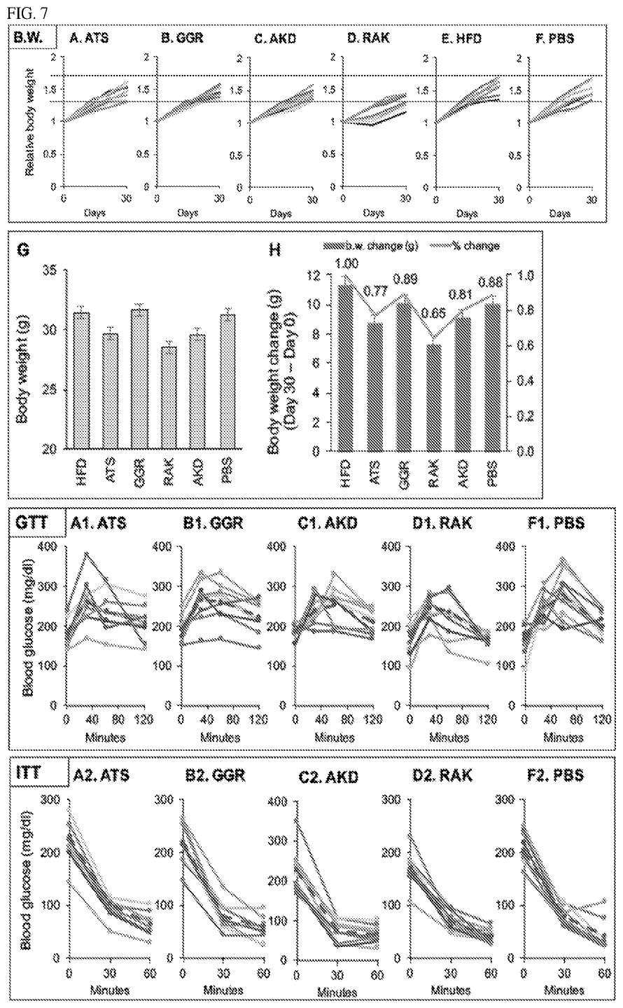

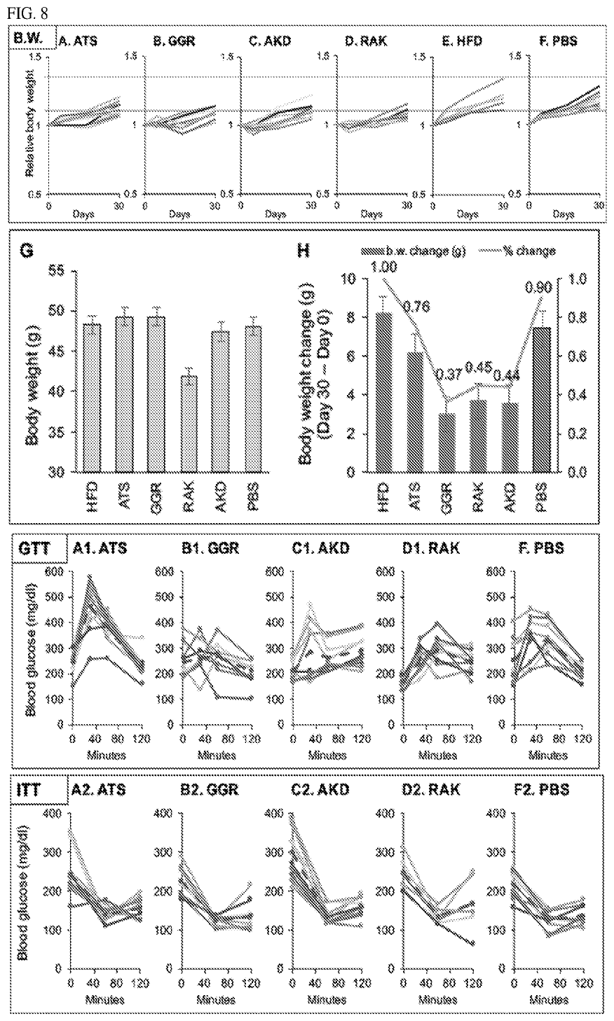

Through the IP and SC studies, it was found that scrambled RKG peptide and vehicle (PBS) had no anti-obesity effects regardless of injection route or schedule. GGR, RAK, and AKD peptides were tested in OPM and OM mice by oral administration. FIGS. 7 and 8 demonstrate that RAK has anti-obesity efficacy in both models and GGR should be further validated. Histological analysis of the fat pads verifies that RAK is more active than GGR. In obesity progression, adipocytes increase in both number and size due to escalating lipid accumulation (Parlee, S. D., et al., Methods Enzymol 537, 93-122, (2014)). Using the methods described above, abdominal fat pads of GGR, RAK, AKD, or PBS-treated DIO mice were histologically analyzed. Epididymal fat tissues were harvested from two mice per experimental group. All samples were fixed in 4% paraformaldehyde, H&E-stained, and imaged. At a 10× magnification, adipocyte cell counting was performed on the digital slide images using an automated adipocyte counting macro with the ImageJ software. A default thresholding method was used and [cell] sizing boundaries of 40-40,000 were set. Limitations of the segmentation tool led to some erroneous selections or regions of interest (ROI), which were manually corrected by adding or erasing lines. The number of cells per representative field were then determined using the ROI manager in ImageJ. Two or three high power fields (hpf) per tissue and two tissue samples per group were analyzed to reduce variability. Average cell count for each group was calculated accordingly. Areas of all adipocytes of interest were measured using the ROI measurement tool (ImageJ) and average area per cell was calculated accordingly.

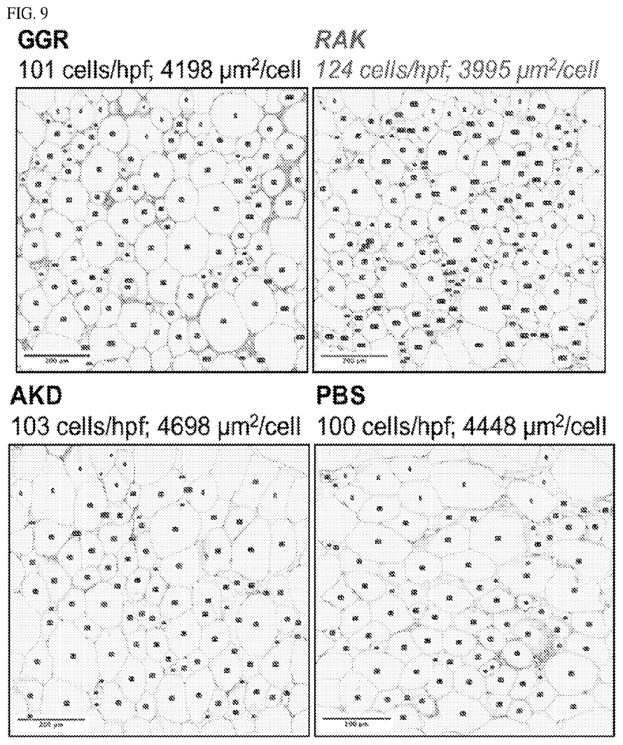

The RAK group was found to have the highest average cell count per field resulting in the lowest average adipocyte area per cell among all experimental groups (FIG. 9). The primary function of adipocytes is to store energy in the form of lipids. The volume of lipid stored in an adipocyte cell increases as the third power of the diameter. Because mature adipocytes are composed of large lipid droplets that are surrounded by very thin cytoplasmic layers, the increase in volume of an adipocyte cell likely indicates increased lipid storage. Thus, smaller adipocyte size/area translates to reduced lipid accumulation. The results show that RAK demonstrates the highest efficacy in this context.

Genetic or dietary models are well-established preclinical models of obesity being used in researches and drug discoveries (Barrett, P., et al., Dis Model Mech 9, 1245-1255, (2016)). Genetic models, including spontaneous mutant and transgenic animals, have been used to define the mechanism of obesity progression (Zhang, Y. et al. Nature 372, 425-432, (1994); Huszar, D. et al. Cell 88, 131-141 (1997)). Since multiple genes contribute to the development of human obesity, obesity is considered polygenic rather than monogenic in nature. Traditional genetic models are now considered as useful mouse models to understand obesity because these monogenic models do not reflect the diversity of human diseases. Dietary models have been advanced to develop polygenic DIO models that better mimic the complex process of human obesity. A DIO model needs fine-tuning, such as dietary choices and meal-type feeding/drinking regimes, to better simulate the heterogeneity of human obesity, an aspect that is not easily mimicked in experimental animals. Recent advances in the development of pre-clinical models of obesity support the use of models that represent the outcome of gene/environment interaction in order to minimize potential artifacts caused by behavioral changes (Barrett et al., supra). The peptides are investigated in 3 pre-clinical models of obesity with 2-3 conditions.

The peptides are further validated in preclinical DIO models. Recent studies have shown the advances in DIO models and the advantages in using them over genetic models (Barrett et al., supra). A DIO model in which there is a defined (high-fat, 60%) diet administered to the mice is used. Some models use variations in fat and sugar contents to assess dietary preference and temporal traits of individual mice to determine the outcomes of interventions such as those described below.

A DIO model generation scheme and injection schedule is shown below:

Obesity with lifestyle change model: Continuous feeding of HFD may be too extreme and does not accurately reflect the life-style changes of a patient with obesity. Obese mice are generated via HFD; once obese, the HFD is changed to a regular diet (Tekland NIH-07) and treatment begins.

Meal-feeding model: Having unlimited (ad libitum) access to a high-energy diet does not translate well to human feeding behavior. Thus, an alternative model is providing set meals at specific times, which more closely imitates human dietary intake. A meal-feeding regime based on past studies and test is used to demonstrate HFD-induced weight gain most accurately.

Binge-type feeding model: Another human feeding behavior to consider is binge eating, which is characterized by overconsumption and lack of control. In a binge-type feeding intervention, animals have ad libitum access to HFD for 2 hours and regular diet for the remaining 22 hours of the day. This model is expected to increase body weight and fat mass in animals, having especially pronounced effects on C57BL/6 mice29. This model is used to observe the effects of extreme meal feeding on obesity progression and to assess the efficacy of the peptides in this particular context of weight gain.

Behavioral phenotyping is performed in DIO models. It is important to assess behavioral phenotypes in obesity models due to increasing evidence that early-life nutritional experiences and epigenetic gene regulation can influence diet choice later in life (Barrett et al., supra; Brenseke, B. et al. Endocrinology 156, 182-192, (2015)). With careful monitoring of specific dietary intake across time periods, it is possible to see which diets are preferred and when, given that the mice have a choice. In some embodiments, computerized systems such as the Comprehensive Lab Animal Monitoring System (Columbus Instruments) or PhenoMaster (TSE Systems) are used to measure food consumption using minute-by minute intervals. With such feeding data, it is possible to gather more information about DIO mice in terms of dietary patterns and preferences and the reasons for variations that we may observe. For example, exposure of obesity-inducing diets to otherwise normal mice may lead to an unpredictable course of weight gain, thus indicating susceptibility or resistance to DIO. Because obesity is a polygenic disease, different diets and feeding regimes are utilized to manipulate genetic models and in turn study environmental and genetic influences on obesity (Barrett et al., supra).

Metabolic changes are monitored. A Micro-Oxymax system (Columbus Instruments) is used for indirect calorimetric (IC) measurements (Matoba, K. et al. Cell Rep 21, 3129-3140, (2017)). Metabolic chambers are used to measure VO2 and VCO2 in individual mice. Respiratory exchange ratio (RER) is calculated to quantify energy expenditure. All data is analyzed using 2-way ANOVA.

Three-week old male C57BL/6J mice are purchased from Jackson Labs. The negative control group is assigned to mice on a normal, low-fat diet (Tekland NIH-07) with no treatment. The positive control group is mice that are fed HFD (DIO Tekland Rodent Diet TD.06414) purchased from Envigo. A group of mice (n=5) on a normal diet are tested with peptide treatment to confirm that the peptides mechanism of action is not relevant in normal mice. The vehicle control group is fed the HFD and treated solely with the vehicle (PBS). Scrambled RKG is omitted because this peptide has no effect over PBS. The DIO treatment cohorts includes: 1) GGR and 2) RAK. Prohibitin mAb is used as a positive control in response to these cohorts (Invitrogen, MA5-12858). A sample size of 10 mice per group is be used unless specified otherwise. This experiment is repeated up to 3 times, hence a total of 2,400 mice-5 models (2 models require both OPM and OM)×10 mice×8 groups×3 repeats×2 dosages are needed to complete the study. Female mice are added to the study to account for possible gender-specific differences and effects in the mouse population in obese conditions.

Peptide formulations are made at concentrations of 60 μM. The mice are orally treated at a dosage of 0.01 mg/g/day or 0.02 mg/g/day three times per week. The dose is calculated specifically for each peptide according to their molecular weights (GGR=288 g/mol, RAK=374 g/mol). Due to the challenge of developing oral delivery systems for mAbs, which are prone to enzymatic degradation and unfolding in the GI tract, a parenteral injection route (subcutaneous) is used to administer prohibitin mAb (Awwad, S. & Angkawinitwong, U. Pharmaceutics 10, (2018)). The dose is calculated according to the mAb molecular weight. HFD feeding and treatment to OPM mice is initiated when their body weights reach approximately 20 g. For OM mice, DIO mice are assigned to the treatment group when their body weights approximately reach 42 g. Oral feeding is facilitated with the use of animal feeding needles (Fisher Scientific) on 1-ml BD syringes. Body weight is measured weekly for a longer term. Real-time live imaging to measure abdominal fat mass provides direct evidence for body weight loss as a consequence of reduced fat. Adipocyte tissue masses are assessed by Micro-CT using a Siemens Inveon Micro-CT scanner. Epididymal fat tissues are imaged on the Micro-CT scanner using CT contrast agents. By using this system and its built-in analytic software, it is possible to calculate fat masses (Wu, C. et al. J Clin Invest 127, 4118-4123, (2017)).

Glucose and insulin tolerance tests are performed in order to characterize the metabolic phenotype of mice and to gain a better understanding of the pathogenesis of obesity (Nagy, C. & Einwallner, E. J Vis Exp, (2018)). This test is used to evaluate the ability to regulate glucose metabolism. Initial blood glucose levels are measured at 16 hours post-fasting using a Care Touch diabetes testing kit. Glucose (3 g/kg body weight) is injected intraperitoneally to each mouse. Blood samples are collected at specific time points: 0, 30, 60, and 120 minutes post-injection. ITT is conducted to monitor whole-body insulin action. GTT and ITT is not performed on the same day. Initial blood glucose levels are measured at 6 hours post-fasting. Insulin (0.75 U/kg body weight) is injected intraperitoneally. Blood samples are collected over time.

It is important to measure lipid profile and obesity-related proteins in blood because reduced lipid uptake by adipocytes may affect these factors. Plasma is separated from all samples and submitted to a medical diagnostic laboratory for plasma lipid profile measurements. Total cholesterol and triglyceride contents is analyzed. Adipokine levels are measured using the Proteome Profiler Mouse Adipokine Array Kit (R&D Systems, ARY103). This analysis detects the levels of obesity related proteins, such as adiponectin, leptin, and TNF-α, in individual samples. It is contemplated that DIO mice with no treatment will have low plasma concentrations of adiponectin and higher-than-normal adipokine levels because adipokines, except for adiponectin, are upregulated in obesity (Inadera, H. Int J Med Sci 5, 248-262 (2008)). Measurement of adipokine levels are useful to assess the degree of pathogenesis in the DIO mice. Cholesterol, triglyceride, and free fatty acid levels in blood may change upon treatment. Total cholesterol, free cholesterol, triglyceride and fatty acid contents, and phospholipid levels are measured via plasma or sera samples using colorimetric enzymatic assays. All assays are performed according to manufacturer instructions from Wako Diagnostics. Free cholesterol in serum is quantitatively determined using the Free Cholesterol E assay (Wako Diagnostics, 993-02501) while total cholesterol is measured with Cholesterol E assay (Wako, 999-02601). Fatty acid contents is measured using the HR Series NEFAHR (2) (Wako, 999-34691, 991-34891, 993-35191, 276-76491). Triglyceride levels are measured with the LType Triglyceride M Assay (Wako, 464-01601).

Immunohistochemistry, proteomics analysis, and biodistribution studies to confirm that the changes in adipocytes are due to peptide effects. Histological analysis of fat pads is performed according to the procedure detailed above. In this study, fat tissues from multiple sites and an increased number of mice are analyzed to statistically generalize prior findings. In addition, the peptide is fluorescently- or radio-labeled for organ distribution. A proteomics service is used to detect expression levels of proteins in adipose tissue samples extracted from mice post-treatment. A differential proteomic analysis allows for analysis of adipose tissue, a heterogeneous tissue with a composition spectrum that includes white and brown Adipocytes (Shields, K. J. & Wu, C. Methods Mol Biol 1788, 243-250, (2018)).

The therapeutic lead peptide is tested in prohibitin knockout (KO) mice to confirm that this peptide exerts its activity in the correct molecular target. It is expected that prohibitin KO mice do not respond to the peptide. To counter the knockout model, a transgenic mouse (Mito-Ob) that overexpresses prohibitin in adipocytes is also developed. It is contemplated that Mito-Ob mice develop obesity independent of diet and the therapeutic lead peptide interferes with obesity progression in Mito-Ob mice. The efficacy of the potential therapeutic lead peptide is compared with current therapies. Three recently commercialized drugs are tested on the DIO mouse models with the same two-phase oral administration schedule used for peptide treatments. Body weight assessments, metabolic profiles, and histological images are analyzed to compare the efficacy of the therapies to that of the peptides.

A one-way analysis of variance (ANOVA) with Dunnetts post hoc is used to compare multiple groups (various peptides/treatments and concentrations) with respect to the control cohorts. Peptide treatment effects on body weight change in DIO mice are analyzed using a repeated measures two way ANOVA with Tukeys post hoc test. All statistical analyses are performed using GraphPad Prism.

### Example 3

### Absorption, Distribution, Metabolism, Excretion (ADME), and Toxicity Properties of Peptides

In vivo absorption, distribution, metabolism, and excretion of the lead RAK peptide is determined in a mouse model. In vitro toxicity screening is also performed. In order to detect absorption, in vivo experiments by oral administration (gavage) as compared to an intravenous (i.v.) administration to calculate bioavailability (F=AUCp.o./AUCi.v.×100) are performed. Administration of the therapeutic lead peptide at 3 doses (0.1 mg/kg, 1 mg/kg, and 10 mg/kg) by oral gavage in C57BL/6J mice (male and female) is compared to an i.v. dose of 1 mg/kg (tail vein injection) in order to calculate the bioavailability. Terminal serum-draws takes place at times 1, 5, 10, 15, 30, 60, 120 and 240 minutes post-administration in separate animals (n=3 for each time point, n=144 males and n=144 females) and levels of the peptide are determined from the samples using HPLC-MS/MS analysis. Area under the curve (AUC) graphs are generated and used to calculate bioavailability (F). If serum levels are detectable at the 240-minute time point, further time points are measured. Vehicle and serum/plasma stabilities are also performed to determine the shelf-life stability of the peptide in solution at room temperature. Measurements over time (5, 15, 30 mins, 1, 2, 4, 6, 8, 12, 24, 48, and 72 hrs) are performed using HPLC-MS/MS.

The volume of distribution is calculated based on the following formula: Vd=dose given (mg)/plasma concentration (mg/L). This is based on a first order kinetics and extrapolation of the kinetic curve after i.v. and oral gavage dosing. Plasma concentrations from absorption studies above are used to determine the plasma concentration at time zero as an extrapolation during the kinetic phase.

In order to determine the clearance of the lead peptide, mouse metabolic cages are utilized to capture all urine after dosing with expectations that the majority of the parent compound and metabolites are be removed via the kidneys. Urine is analyzed for the parent compound and projected metabolites (individual amino acids) using HPLC-MS/MS. Detection for the glucuronidation of the peptide is also measured. Clearance is calculated using the following formula: CL=rate of elimination (mg/h)/plasma concentration (mg/L). The half-life of the peptide is calculated based on the following formula: t1/2=0.693×Vd/CL. Feces are also collected if the parent peptide and metabolites are insufficient to extrapolate back to 95-99% of the initial dose. In previous studies, the majority (>99%) of peptide administered into the GI tract is absorbed and does not re-enter the hepatic portal system.

WinNonlinR (Pharsight Co., CA, USA) is used to quantify and simulate predictive PK/PD parameters as compared to observed values. The simulation PK/PD parameters are Tmax, Cmax, ke, t1/2 and AUC.

Initial metabolism and toxicology of the peptide is calculated through In Vitro ADMET Laboratories (IVAL) toxicity tests for hepatotoxicity screens, p450 inhibition, CYP induction, cytotoxicity, Ames activity, and hERG testing (Charles River). In addition, the lungs, heart, liver, kidneys, spleen and brain are removed and weighed to determine differences between peptide vs. vehicle-treated animals. Also, fat tissues are fixed and H&E stained for histopathology to compare peptide-treated animals to control and vehicle-treated animals for the 120- and 240-minute time points.

Design-ExpertR (Stat-Ease, Minn., MN) software is used to design experiments and to conduct the multivariate statistical analyses of the data. All data is analyzed using the statistical software packages, SigmaPlotR/SigmaStatR (JandelSci.). Control group=1, naive group (n=3), and 1 vehicle-only (n=3) administration injected group, two routes of administration, for 8 time-points (3 mice/group×2 genders×4 doses (3 peptide+1 vehicle)×2 routes of administration×8 time-points+3 naive=387 mice in total). Experimenters are blinded to treatment and control groups, giving 80% power to detect a treatment effect size of 20% compared to a baseline response of 5% at a significance level of 0.05 (Clayton, J. A. & Collins, F. S. Nature 509, 282-283 (2014); Andrews, N. A. et al. Pain 157, 901-909, (2016)). Numbers required to achieve statistical power for the ADME studies were determined by G.Power3.1.

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A pharmaceutical, dietary supplement, or nutraceutical composition comprising one or more peptides having an amino acid sequence selected from the group consisting of KGGRAKD (SEQ ID NO:1), KGG, GRA, GGR, RAK, AKD, DKA, KAR, RGG, ARG, GGK, and DKARGGK (SEQ ID NO:2) or a variant or mimetic thereof.

2. The composition of claim 1, wherein said peptide is cyclized.

3. The composition of claim 2, wherein said peptide is cyclized via the addition of a cysteine to each end of said peptide.

4. The composition of claim 1, wherein said peptide is modified.

5. The composition of claim 1, wherein said composition comprises a pharmaceutically-acceptable carrier.

6. The composition of claim 5, further comprising one or more additional therapeutic agents.

7. The composition of claim 1, wherein said composition is formulated for oral delivery, local injection, or systemic injection.

8. The composition of claim 1, wherein said composition comprises two or more of said peptides.

9. The composition of claim 1, wherein said supplement is a beverage.

10. The composition of claim 1, wherein said composition is formulated as a capsule, tablet, or powder.

11. The composition of claim 1, wherein said composition is formulated as a food product.

12-14. (canceled)

15. A method of treating or preventing obesity, comprising:
administering the composition of claim 1 to a subject in need thereof.

16. The method of claim 15, wherein said administering decreases fat storage and/or promotes weight loss in said subject.

17. The method of claim 15, wherein said subject is overweight or obese.
